(19) United States
(12) Patent Application Publication
(10) Pub. No.: US 2022/0257221 A1
(43) Pub. Date: Aug. 18, 2022

(54) High Payload Biopsy Needles And Methods

(71) Applicant: NXT Biomedical, LLC, Irvine, CA (US)

(72) Inventors: Stanton J. Rowe, Irvine, CA (US); Robert S. Schwartz, Irvine, CA (US); Elliot Howard, Irvine, CA (US); Robert C. Taft, Irvine, CA (US); Alexander Siegel, Irvine, CA (US); Joseph Passman, Irvine, CA (US); Glen Rabito, Irvine, CA (US); Abubaker Khalifa, Irvine, CA (US)

(73) Assignee: NXT Biomedical, LLC, Irvine, CA (US)

(21) Appl. No.: 17/597,857

(22) PCT Filed: Jul. 29, 2020
(86) PCT No.: PCT/US20/44066

§ 371 Date: Jan. 26, 2022

Related U.S. Application Data
(60) Provisional application No. 62/880,037, filed on Jul. 29, 2019.
(60) Provisional application No. 62/932,416, filed on Nov. 7, 2019.

(51) Int. Cl. A61B 10/02; A61B 10/04

(52) U.S. Cl. CPC A61B 10/0266; A61B 2010/045; A61B 10/04

(57) ABSTRACT

Low profile biopsy tools and methods that allow relatively large biopsy samples to be taken. The biopsy tools have a first configuration that allows for atraumatic navigation to a targeted lesion, and a second configuration in which a distal end of the biopsy tool expands to take a large tissue sample. The tissue sample may then be retracted into a sheath or other containment feature and removed at a reduced diameter. Also included is an anchored guidewire usable to practice method for navigating a biopsy tool to a targeted lesion after an endoscope has been removed.

### RELATED APPLICATIONS

This application claims benefit of and priority to U.S. Provisional Application Ser. No. 62/880,037 filed Jul. 29, 2019 entitled High Payload Biopsy Needles, and to U.S. Provisional Application Ser. No. 62/932,416, filed Nov. 7, 2019 entitled High Payload Biopsy Needles-2.0, both of which are hereby incorporated herein by reference in their entireties.

### BACKGROUND OF THE INVENTION

Biopsy procedures involve the removal of a tissue sample in order to conduct tests and analyses on the tissue for diagnostic purposes. Some biopsies, such as lung biopsies, as well as numerous others, are performed using Fine Needle Aspiration Biopsy (FNAB) needles or Core Needle Biopsy (CNB) biopsy tools. The FNAB's are smaller in profile and therefore carry fewer procedural risks and complications. However, FNAB needles tends to have a low test sensitivity and a high specificity, meaning that if a patient tests positive for something, such as cancer, there is a high probability that they have cancer, but if that patient tests negative for cancer, there is a high probability that the test result cannot be trusted and another test would have to be performed. In other words, FNAB's tend to have a high percentage of false negatives due to their small sample sizes.

Conversely, CNB's take larger samples and therefore have higher sensitivities, resulting in fewer false negatives. However, the much larger profile of a CNB is associated with more procedural risks and potential complications to patients.

As such, there is a need for a biopsy needle that combines the low-profile of an FNAB and the biopsy payload of a CNB.

### OBJECTS AND SUMMARY OF THE INVENTION

The various inventions described herein are directed to biopsy tool designs and methods that have a low-profile but are able to obtain large enough sample sizes to obviate the need for further sampling if a negative test result occurs from a first sample.

At least one aspect of the invention provides a biopsy tool having at least two configurations: a first configuration in which the biopsy tool is constrained and has a relatively constant diameter, and resembles an FNAB needle, and a second or deployed configuration in which at least a distal portion of the tool expands into a tissue-extracting configuration, allowing the tool to gather a greater amount of tissue than a similarly-sized device that does not expand from a first configuration.

One aspect of the invention is a biopsy tool comprising a sheath, a needle disposed within the sheath and having a first configuration and a second configuration, wherein in the first configuration the needle is radially constrained by the sheath, wherein in the second configuration the needle includes a tissue engaging feature that may expand such that the tissue engaging feature is located radially outside an outer diameter of the sheath, wherein further in the second configuration the tissue engaging features is useable to at least partially remove tissue samples and relocate the tissue samples to an inner lumen of the biopsy tool.

Another aspect of the invention is a tissue removal method comprising navigating a sheath to a target location, advancing a tool having a first configuration while housed within the sheath until at least a distal end of the tool emerges from a distal end of the sheath, changing a state of the tool from said first configuration to a second configuration, said second configuration having a width that is greater than a diameter of the sheath, using the tool in the second state to remove tissue from the target location, changing the state of the tool from the second configuration back to the first configuration, and retracting the tool into the sheath with the removed tissue.

Yet another aspect of the invention is a method of taking a biopsy comprising navigating an endoscope having a working channel to a location adjacent a targeted tissue area, advancing a distal end of the guidewire out of a distal end of the working channel and into the targeted tissue area, deploying an anchor at the distal end of the guidewire, pulling the endoscope out of the patient while leaving the guidewire in place, advancing a biopsy tool over the guidewire, capturing a tissue sample using the biopsy tool, and removing the tissue sample, the biopsy tool, and the guidewire from the patient.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a cutaway view of an embodiment of a tool of the invention;

FIG. 2 is a cutaway view of an embodiment of a tool of the invention;

FIG. 3 is a cutaway view of an embodiment of a tool of the invention;

FIG. 4A is a perspective view of an embodiment of a tool of the invention;

FIG. 4B is a perspective view of an embodiment of a tool of the invention;

FIG. 5 is a plan view of an embodiment of a tool of the invention;

FIG. 6 is a plan view of an embodiment of a tool of the invention;

FIG. 7 is a perspective view of an embodiment of a tool of the invention;

FIG. 8 is a perspective view of an embodiment of a tool of the invention;

FIG. 9 is a perspective view of an embodiment of a tool of the invention;

FIG. 10 is a side view of an embodiment of a tool of the invention;

FIG. 11 is a side view of an embodiment of a tool of the invention;

FIG. 12 is a cutaway view of an embodiment of a tool of the invention;

FIG. 13 is a side view of an embodiment of a tool of the invention;

FIG. 14 is a side view of an embodiment of a tool of the invention;

FIG. 15 is a cutaway view of an embodiment of a tool of the invention;

FIG. 16 is a perspective view of an embodiment of a tool of the invention;

FIG. 17 is a side view of an embodiment of a tool of the invention;

FIG. 18 is a cutaway view of an embodiment of a tool of the invention;

FIG. 19A is a side view of an embodiment of a tool of the invention;

FIG. 19B is a side view of an embodiment of a tool of the invention;

FIG. 19C is a side view of an embodiment of a tool of the invention;

FIG. 20 is a perspective view of an embodiment of a tool of the invention;

FIG. 21 is a cutaway view of an embodiment of a tool of the invention;

FIG. 22 is a perspective view of an embodiment of a tool of the invention;

FIG. 23 is a perspective view of an embodiment of a tool of the invention;

FIG. 24 is a perspective view of an embodiment of a tool of the invention;

FIG. 25 is a cutaway view of an embodiment of a tool of the invention;

FIG. 26 is a perspective view of an embodiment of a tool of the invention;

FIG. 27 is a perspective view of an embodiment of a tool of the invention;

FIG. 28 is a perspective view of an embodiment of a tool of the invention;

FIG. 29A is a perspective view of an embodiment of a tool of the invention;

FIG. 29B is a cutaway view of an embodiment of a tool of the invention;

FIG. 30 is a side view of an embodiment of a tool of the invention;

FIG. 31 is a side view of an embodiment of a tool of the invention;

FIG. 32 is a side view of an embodiment of a tool of the invention;

FIG. 33 is a side view of an embodiment of a tool of the invention;

FIG. 34 is a cutaway view of an embodiment of a tool of the invention;

FIG. 35 is a side view of an embodiment of a tool of the invention;

FIG. 36 is a side view of an embodiment of a tool of the invention;

FIG. 37 is a side view of an embodiment of a tool of the invention in a deployed state;

FIG. 38 is a side view of the embodiment of FIG. 38 being retracted;

FIG. 39 is a side view of an embodiment of a tool of the invention in a deployed state; and,

FIG. 40 is a side view of the embodiment of FIG. 39 being retracted.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

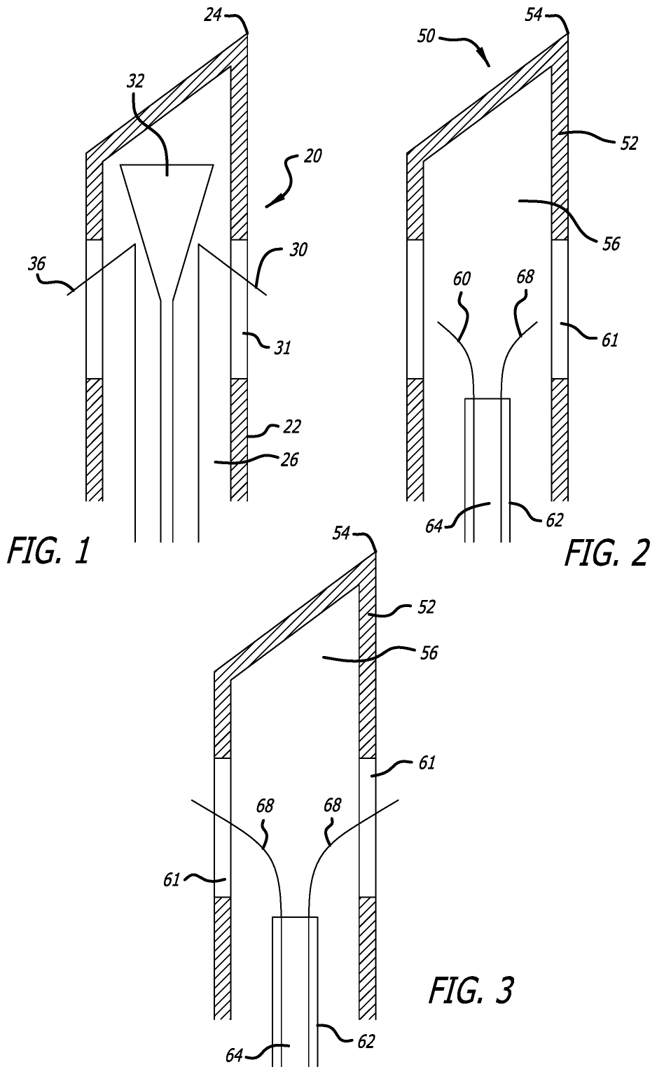

Referring now to the Figures and first to FIG. 1, there is shown an embodiment 20 of a biopsy device that includes a needle-like body 22 with a sharpened distal end 24. The body 22 has an internal lumen 26 in which are housed one or more grasping members 30. The grasping members 30 may be inwardly biased and splayed outwardly with a mechanism 32. In one embodiment, the mechanism 32 resides distal of the grasping members and is shaped such that, upon proximal retraction of the mechanism 32, the grasping members 30 are forced outwardly. The grasping mechanisms may include tissue grabbing features 36, such as hooks, that pull tissue samples from the surrounding tissue. This effect may be amplified by pulling the entire device proximally, thereby dragging the grabbing features 36 through the tissue.

The grasping members 30 may be disposed adjacent to longitudinal slots 31 in the needle-like body 22. Thus, the grasping members 30 have radial freedom to radiate or splay from the body 22 when the mechanism 32 is retracted. In the embodiment involving inwardly biased grasping members 30, the proximal ends of the grasping mechanisms slope inwardly, thus leaving a space between the proximal end of the slot and the grasping mechanism that can be used to capture and retain the tissue samples. In at least one embodiment, a suction may be applied to the lumen 26 to increase the amount of tissue obtained.

In a similar embodiment 50, shown in FIG. 2, there is a biopsy device that includes a needle-like body 52 with a sharpened distal end 54. The body 52 has an internal lumen 56 in which are housed one or more grasping members 60. The grasping members 60 may be outwardly biased and contained with an inner sheath 62. In one embodiment, the sheath 62 includes an inner lumen 64 in which the grasping members reside. Upon proximal retraction of the sheath 62, the grasping members 60 naturally splay outwardly. The grasping mechanisms may include tissue grabbing features 66, such as hooks, that pull tissue samples from the surrounding tissue, similar to those of FIG. 1. Alternatively, such as is shown in FIG. 2, the grasping mechanisms may be straight and have sharpened ends 68.

The grasping members 60 may disposed adjacent to longitudinal slots 61 in the needle-like body 52. Thus, the grasping members 60 have radial freedom to radiate or splay from the body 52 when the sheath 62 is retracted and the distal ends 68 are released. Thus, when the sheath 62 is retracted, such as is shown in FIG. 3, the grasping members 60 are released through the slots and engage the surrounding tissue. Tissue samples may be obtained by pushing the device 50 in a distal direction, thus allowing the sharpened ends 68 to chisel tissue samples from the surrounding tissues, which are then captured within the slots 61. In at least one embodiment, a suction may be applied to the lumen 56 or 64 to increase the amount of tissue obtained.

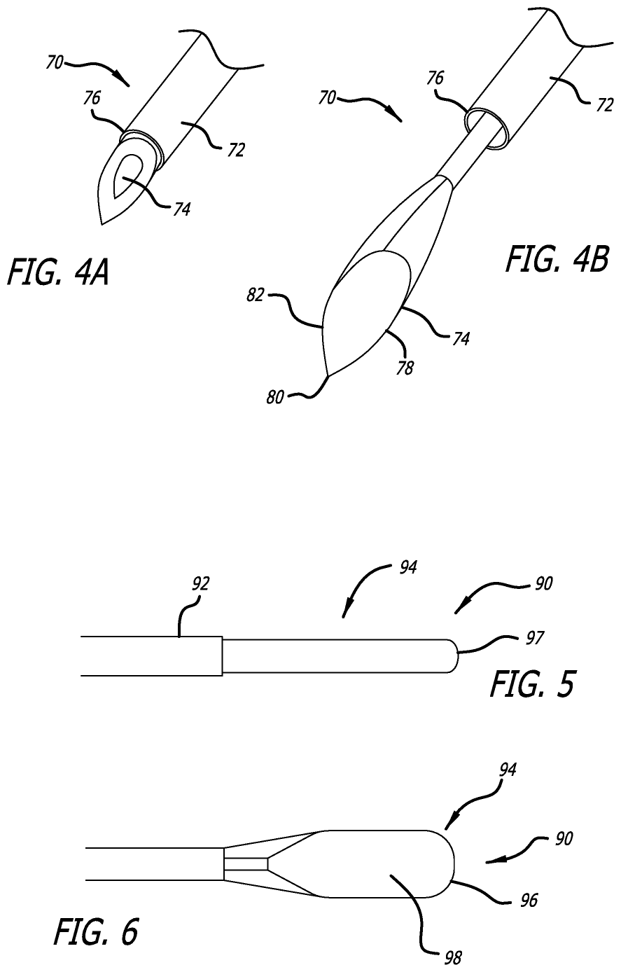

FIGS. 4A and 4B show an embodiment 70 of a biopsy device that includes an outer sheath 72 containing an expandable needle 74. FIG. 4A shows the embodiment 70 in a first configuration prior to expansion and FIG. 4B shows the embodiment 70 in a second, expanded configuration. The expandable needle, in at least one embodiment, is formed from a rolled sheet of metal, such as nitinol, stainless steel, or other suitable metal, and is rolled helically or diagonally, such that it is able to expand soon after being pushed distally out of an end 76 of the sheath 72. In at least one embodiment, a leading edge 78 of the needle 74 includes a distal point 80, which allows the needle 74 to penetrate tissue as it is advanced out of the sheath 72. In at least one embodiment, the needle further or alternatively includes one or more sharpened leading edges 82. In the embodiment of FIGS. 4A and 4B, these sharpened edges 82 may be located on either side of the point 80. In other embodiments, the metal sheet is thin enough that the need for a sharpened edge is obviated.

In operation, the device 70 is advanced to a target location with the needle 74 contained within the sheath 72. The sheath may have a leading trocar edge to serve as a needle while navigating to the target location. Alternatively, a guidewire may be used to navigate the device 70 to the target location. Another alternative accomplishes navigation by advancing the needle 74 slightly past the end 76 of the sheath 72 such that the needle is not yet expanded but the sharpened point 80 is exposed and may be used to puncture tissue.

Once the target location is reached, the needle 74 is advanced and self-expands. The entire device 70 is then advanced, thereby cutting and funneling tissue samples into the sheath 72. This may be assisted by applying suction to the sheath 72.

FIGS. 5 and 6 show an embodiment 90 of a biopsy device that includes an outer sheath 92 containing an expandable scoop 94. The expandable scoop 94, in at least one embodiment, is formed from a wire form 96, over which a film 98 is attached. The wire form 96, may be a metallic material, such as nitinol, stainless steel, or other suitable metal, and has resilient or memory properties such that it may be rolled and contained within the sheath 92 until it is advance out of the sheath, at which time the wire form 96, unrolls and forms an open configuration, such as an oval, surrounded on one side by the film 98. The wire form 96 may be sharpened, or thin and strong enough to cut through tissue without sharpening.

In operation, the device 90 is advanced to a target location with the scoop 94 contained within the sheath 92. The sheath 92 may have a leading trocar edge to serve as a needle while navigating to the target location. Alternatively, a guidewire may be used to navigate the device 90 to the target location. Another alternative accomplishes navigation by advancing the scoop 94 slightly past a distal end 97 of the sheath 92 such that the wire form 96 is not yet expanded but acts as a sharpened point and may be used to puncture tissue.

Once the target location is reached, the scoop 94 is advanced and self-expands, as seen in FIG. 6. The scoop 94 is then rotated about a longitudinal axis of the device, thereby cutting tissue samples, which are collected by the film 98. Retracting the scoop 94 back into the sheath 92 causes the scoop to roll or fold and contain the tissue sample while pulling it into the sheath 92, such as in FIG. 5. This embodiment, like all of the embodiments described herein, may use suction applied to the sheath 92 to assist in ensuring the tissue is not inadvertently dropped during the retrieval process.

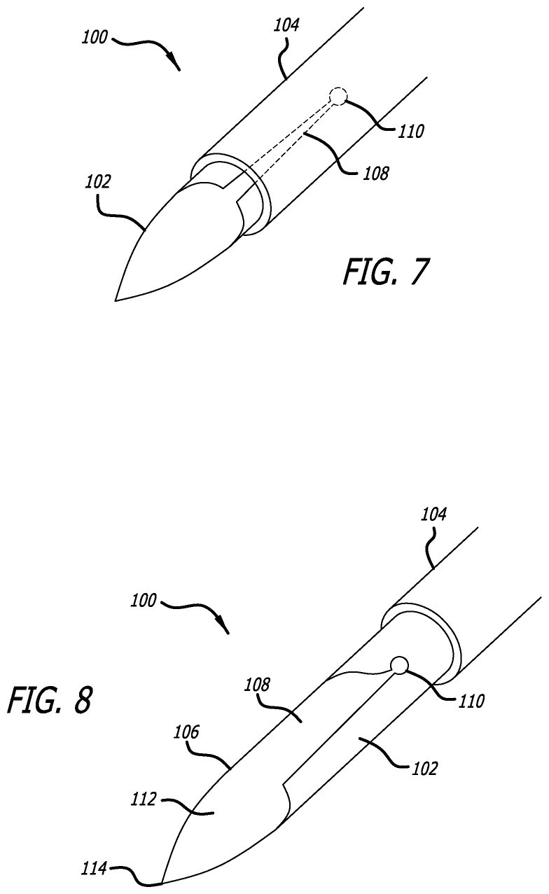

FIGS. 7 and 8 show an embodiment 100 of an expandable biopsy device that includes a cutting tool 102 that is contained within a sheath 104. FIG. 7 shows the device 100 in a first configuration and FIG. 8 shows the device 100 in a second, expanded configuration. The tool 102 is formed similarly to the rolled metal needle 74 of FIG. 4, except that, at least in one embodiment, the tool 102, when expanded, forms a blade 106. The blade 106 is created by forming a slit 108 in the end of the tool 102. The slit 108 may include a feature 110, such as a circular hole, at the proximal end of the slit 108 in order to prevent crack propagation and to allow the blade to expand evenly along its length.

As seen in FIG. 8, the blade 106 has spring-like properties that cause the slit 108 to open, at least slightly, upon emerging distally from the sheath 104. When splayed, the blade 106 radiates somewhat tangentially to the rest of the tool 102. As such, the tool 102 may be rotated to cut tissue surrounding the tool 102. The tissue will thus be scalped and fed into a center lumen 112 of the tool 102. Suction may be used additionally to increase the size of the sample.

Like some previously disclosed embodiments, the tool 102 may include a pointed tip 114 to make advancement through tissue easier. The sheath 104 may alternatively include a pointed tip (not shown). Additionally, the tool 102 is not limited to a single blade. Additional slits 108 may be used to create multiple blades 106.

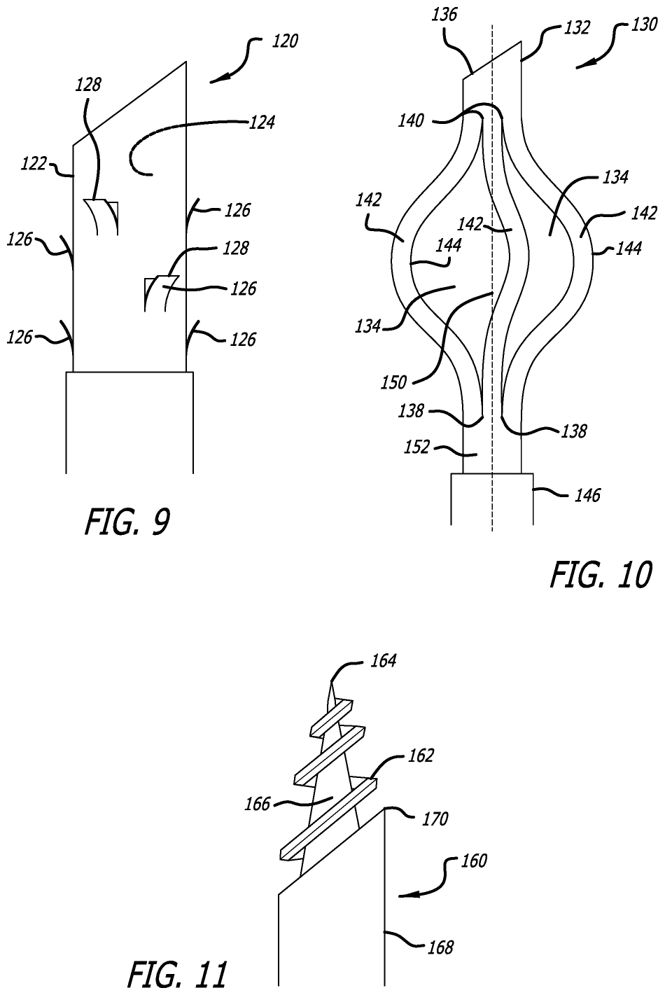

One example of a tool 120 having multiple blades is shown in FIG. 9. The tool 120 has a continuous, thin cylindrical wall 122 that defines a central lumen 124. A plurality of tabs 126 are cut into the walls and are outwardly biased. Thus, when released from a sheath, such as those previously mentioned, the tabs 126 splay outward and have edges 128 that form blades. Rotating the tool 120 through tissue causes the sliced tissue to be directed inwardly to the lumen 124 for collection. Suction may be used to enhance the collection.

FIG. 10 shows an embodiment 130 of a biopsy device that may have more than one blade or cutting edge. This embodiment 130 includes a needle 132 that has a plurality of slits 134 formed near a distal end 136 of the needle 132. The slits 134 have proximal ends 138 and distal ends 140. The distal ends 140 terminate prior to the distal end 136 of the needle 132. The material between the proximal ends 138 and distal ends 140 of the slits 134 splay outwardly to form wings 142 that include cutting edges 144.

In at least one embodiment, the wings 142 are outwardly biased such that, upon release from a sheath 146 or other restraint, the wings 142 passively expand, drawing the distal end 136 of the needle proximally. The act of allowing the wings 142 to expand and then retracting the wings 142 back into the sheath may result in the collection of a sufficient amount of tissue for a desired sample. In at least one embodiment, the distal edge 148 of the sheath is sharpened such that any tissue trapped, but not completely severed, by the wings 142, gets severed by the distal edge 148 of the sheath 146 as the sheath 146 is advanced over the needle 132, in preparation for removal of the device 130 from the patient.

In at least one embodiment, the wings 142 are inwardly biased or biased toward a straight configuration. The wings 142 may then be actively expanded by including a pull wire 150 that runs through the center lumen 152 and is attached to the distal end 136 of the needle 132. Thus, once the needle 132 has been advanced to a target location, the pull wire is retracted relative to the needle 132, causing the wings 142 to splay. This active expansion configuration may obviate the need for an external sheath. Whether active or passive, the wings 142 may be rotated when splayed in order to take larger samples.

FIG. 11 shows an embodiment 160 of a rotating biopsy device that includes an auger blade 162 helically radiating from a distal end 164 of the shaft 166 of the device 160. The device 160 further includes a sheath 168 in which the shaft 166 and auger blade 162 may be contained during navigation and retraction. During use the device 160 is navigated to the target location and the auger blade 162 is extended distally from the sheath 168 and rotated into the target tissue mass. The rotating action of the auger will pull tissue proximally into the sheath 168, which may include a sharpened distal edge 170 that will provide cleanly cut tissue samples.

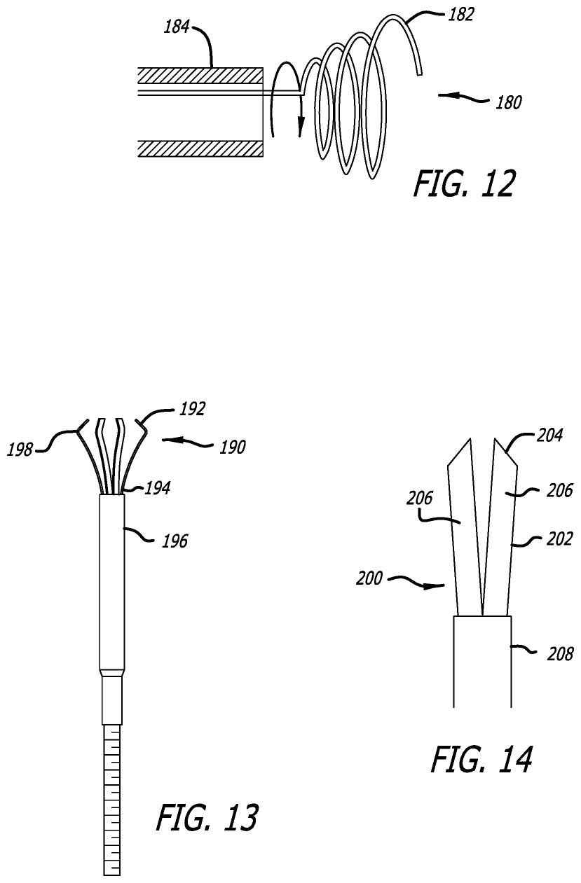

FIG. 12 shows an embodiment 180 of a rotating collection device that is also expandable. The device 180 includes a distally disposed expandable coil 182. The coil is wound helically such that when rotated through tissue, the coil 182 advances distally and expands radially. Tissue is then collected by either retracting the coil 182 into a sheath 184 or advancing the sheath 184 over the coil 182. The distal edge 186 of the sheath 184 may be sharpened to aid in tissue dissection. Suction may also be supplied to assist in tissue retrieval.

FIG. 13 shows an embodiment 190 of a biopsy device that includes extendable and retractable graspers 192 that emerge from a distal end 194 of a sheath 196. Some or all of the individual graspers 192 are shaped to splay outwardly when advanced. Some or all of the individual graspers 192 include hook-like bends 198 near their distal ends, giving the graspers the ability to grip tissue and pull the tissue into the sheath 196. The distal end 194 of the sheath may be sharpened to help dissect tissue from the surrounding area as it is pulled into the sheath 196.

FIG. 14 shows an embodiment 200 of a device having a needle 202 that is split longitudinally at its distal end 204 to form a plurality of arms 206. The arms 206 may be biased outwardly such that as the needle 202 emerges from a sheath 208, the arms passively expand or splay outwardly. Once expanded, the needle 202 may be driven forward to grab a larger amount of tissue than would be possible with a non-expanding needle. The needle 202 may then be resheathed to collapse and capture the tissue. The tissue may be prevented from escaping during the resheathing process by either supplying suction to a center lumen of the needle 202, or by advancing the sheath forward over the needle 202 such that adjacent surrounding tissues act against the tissue being captured, thus preventing the tissue from being ejected by the closing arms 206.

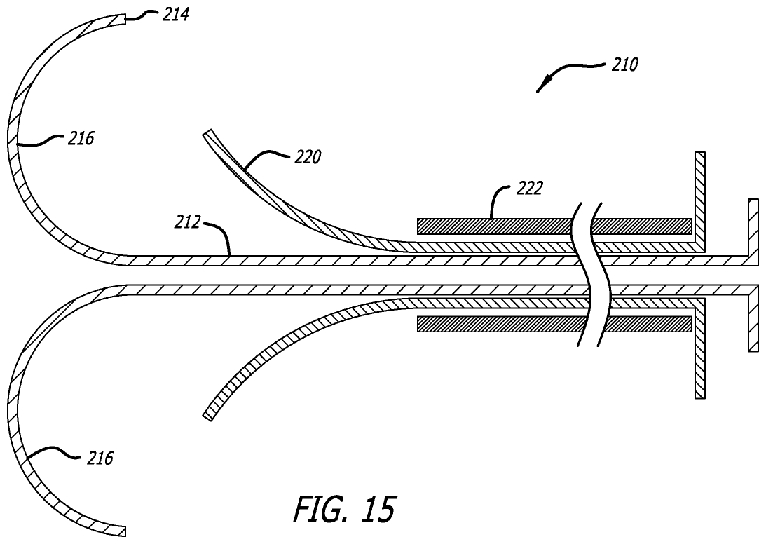

FIG. 15 shows another embodiment 210 of a biopsy device that includes a needle 212 that is split longitudinally at its distal end 214 to form a plurality of arms 216. The arms 216 may be biased outwardly to such an extent that they curl back on themselves when deployed. In doing so, the arms 216 are able to catch tissue using an outside surface of the arms 216. An additional, inner sheath 220 is used that splays outwardly when deployed from the outer sheath 222 to form a funnel into which the tissue is pulled and trapped when the needle 212 is retracted into the device 210. The inner sheath 220 may be a nitinol braided tubular mesh, a rolled sheet such as the needle 74 of FIG. 4, or other expandable funnel designs such as an expandable wire form with a film attached thereto. These are provided as non-limiting examples.

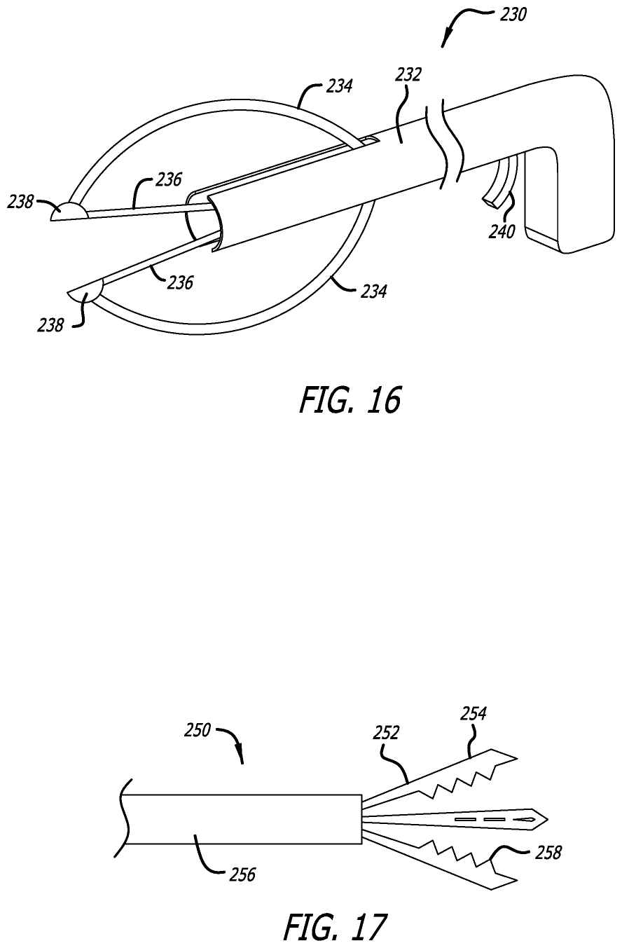

FIG. 16 shows an embodiment 230 of an actively expandable biopsy device that includes a sheath 232 that contains two sets of arms 234 and 236. Each set of arms 234 and 236 are joined together at a distal end, which includes a tissue grasping surface 238. By manipulating the relative longitudinal positions of the sets of arms 234 and 236, the distance between the two grasping surfaces 238 is changed. As such, the device acts as a set of pinchers that are able to grasp tissue. In one embodiment, a trigger mechanism 240 is provided at the proximal end that allows a physician to manipulate the grasping surfaces 238 by squeezing the trigger with one hand. The grasping surfaces 238 may include a wide variety of features such as blades, textured surfaces, opposing blades that act like scissors, and the like.

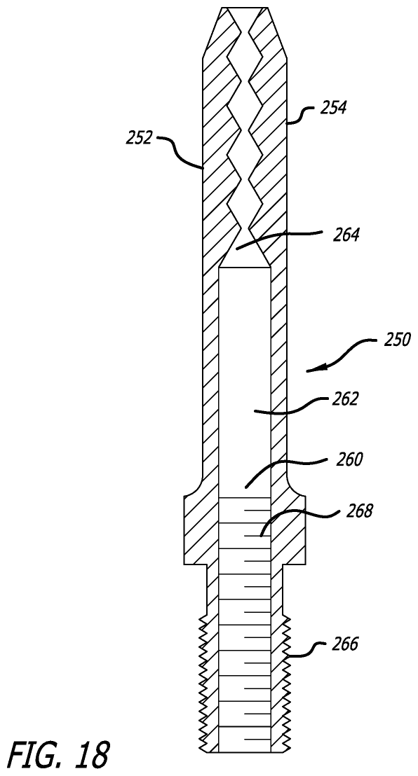

FIG. 17 shows an embodiment 250 of a split needle biopsy tool that includes a split needle 252 having arms 254 formed from making slits in the distal end as previously discussed. The needle 252 is contained within a sheath 256. The arms 254 include inwardly projecting teeth 258 with pointed or sharpened surfaces for macerating tissue. Referring to FIG. 18, there is shown a longitudinal cross-section of the device 250 that shows its inner workings. As can be seen, the split needle 252 includes an inner threaded cavity 260 that houses an activation shaft 262. The shaft 262 has a tapered distal end 264 and the cavity 260 is shaped to match the shaft 262 when in a retracted position, as shown. Rotating the shaft 262 relative to the needle 252 causes external threads 266 on the shaft 262 to engage internal threads 268 formed in the cavity 260 such that longitudinal or axial motion of the shaft 262 relative to the needle 252 results. Advancing the shaft 262 distally causes the tapered distal end 264 of the shaft 262 to force open the arms 254 of the needle 252. Once the arms 254 are deployed, the internal threads 268 end and further rotation of the shaft 262 is translated into rotation of the needle 252. The rotation of the needle 252 causes the teeth 258 of the splayed arms 254 to slice through tissue, which may then be captured by retracting the arms 254 through reverse rotation of the shaft 262.

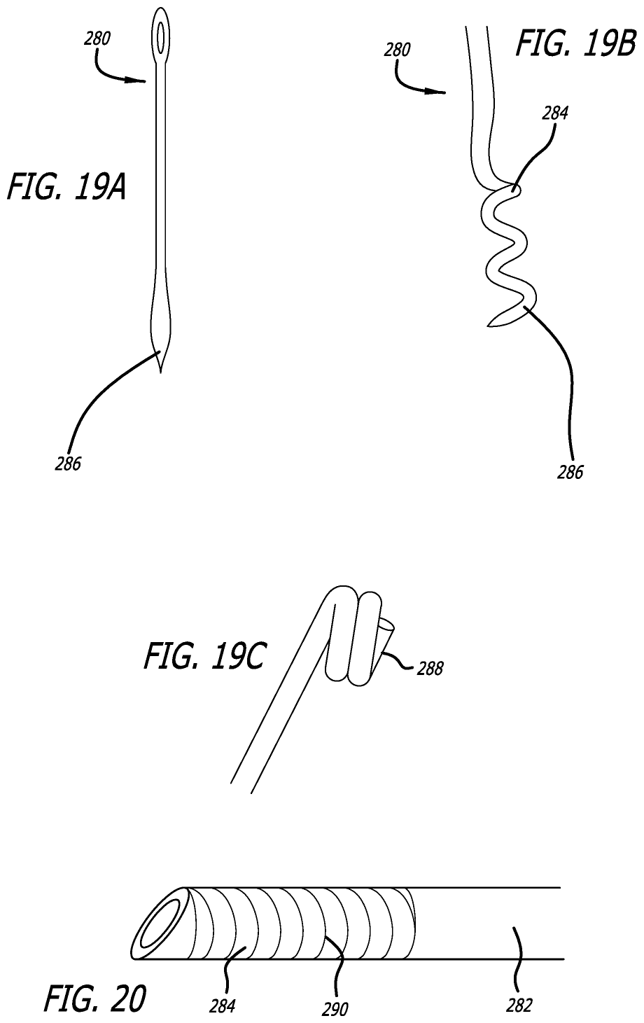

FIG. 19A shows an embodiment 280 of a biopsy device that includes a needle 282 that has a first configuration in which it is straight and assumes a second configuration (FIG. 19B) in which a corkscrew shape 284 forms at a distal end 286 of the needle 282. The needle 282 may include a heat-set nitinol tip such that the needle self-forms into the second configuration upon release from a sheath. FIG. 19C shows an alternative second configuration in which the distal end 286 of the needle 282 forms a spring shape 288.

FIG. 20 shows an example of how to manufacture the needle 282 having a distal end that expands into a corkscrew shape 284. As shown, the needle, which may be hollow or solid, has a helical pattern 290 cut into the distal end with a laser. The needle 282 is then heat-set to an expanded configuration exemplified by the corkscrew shape 284 of FIG. 19B.

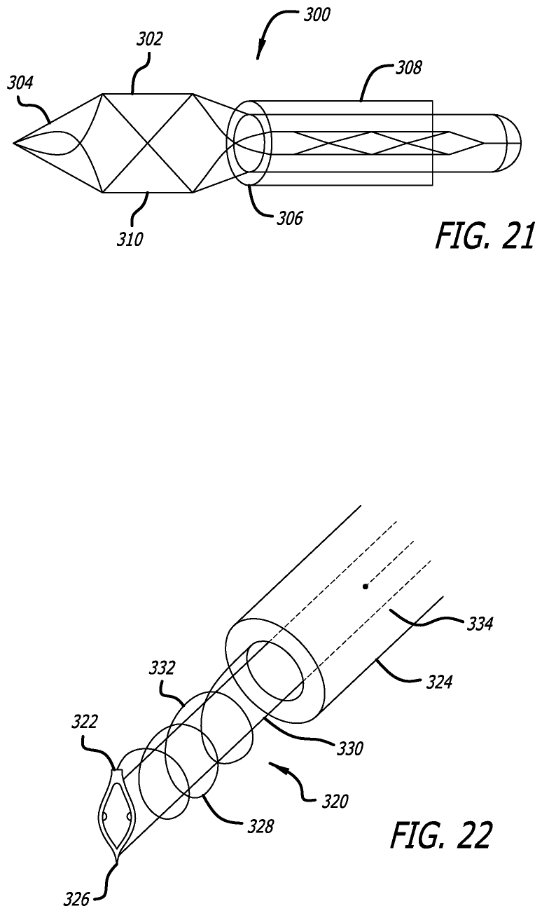

FIG. 21 shows an embodiment 300 of a stent-like biopsy device. Similar to the embodiment 90 of FIGS. 5 and 6 except that the wireform and film combination is replaced by a stent 302 that acts as a needle and a retrieving basket. The stent 302 may be equipped with a sharp distal cutting edge 304 that may be deployed out of the distal end 306 of a sheath 308, upon which it self-expands to retrieve a desired amount of tissue. Upon retraction, the tissue is forced into the sheath 308 by a variety of struts 310 that make up the stent 302. The stent 302 then collapses into the sheath 308, capturing the tissue in the distal end of the sheath.

FIG. 22 depicts an embodiment 320 of a biopsy device that includes an expandable needle 322 contained within a sheath 324. The needle 322 is comprised of a sharpened distal tip 326 and a coil or stent-like expandable body 328. The body 328 has a diameter that decreases when the device is stretched longitudinally and increases when the device returns to a non-stretched state through spring force. The device 320 further includes an inner stylet 330 that runs through a center lumen 332 of the body 328 and attaches to the distal tip 326 of the needle 322.

In use, the sheath 324 is navigated to a target location and the stylet 330 is advanced such that the needle 322 is pulled out of the sheath 324 and through the tissue. Once the stylet 322 is fully advanced, the body 328 expands in the tissue, capturing and dislodging tissue in the process. The needle 322 is then retracted into the sheath 324 and removed with the tissue sample. In at least one embodiment, a pull wire 334 is attached to a proximal end of the needle 322 to aid in retrieving the needle 322. Pulling the proximal end of the needle 322 causes the needle to elongate and narrow in diameter, allowing for an easier retrieval process.

As stated above, any of the embodiments described herein may be aided through the use of suction. Additionally, and of the embodiments described herein may be combined with a high velocity flow of fluid or gas ejected from the distal ends of the devices in order to cut through the tissue, allowing for an easier retrieval and a larger payload. Additionally, different patterns or shapes of tissues can be cut.

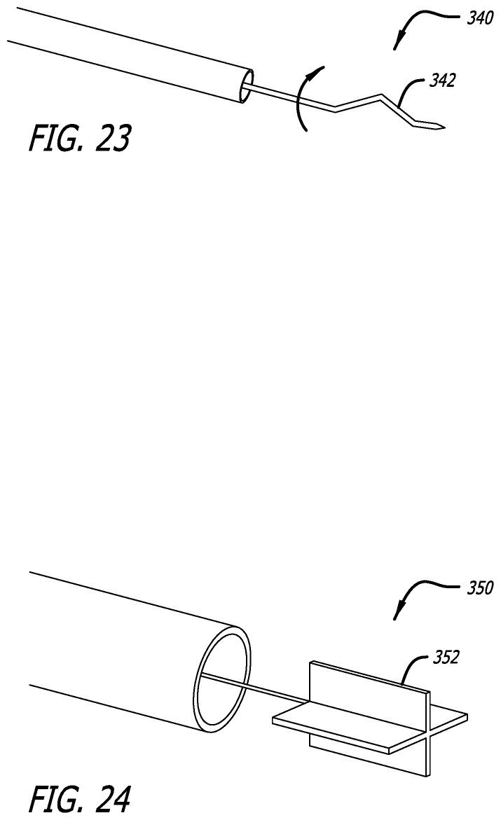

Alternatively or additionally, a wire or bladed wire can be used to supplement any of the embodiments described herein, or with a traditional FNAB needle. FIG. 23 shows an embodiment 340 of such a wire being used with a traditional FNAB needle. The wire 340 may include a bent or pre-shaped distal end 342 that can be inserted through the needle such that when the wire 340 exits the needle, it assumes the bent shape in the target tissue. Once the wire 340 has assumed the bent shape, it may be rotated about a central axis of the wire 340 at a speed appropriate to cut the targeted tissue into smaller pieces so the tissue can be more easily aspirated to extracted mechanically. FIG. 24 shows an embodiment 350 of a wire with one or more expandable blades 352 that may be used in a similar manner. Additionally or alternatively, the wires, such as 340 or 350 may be energized with RF energy in order to more efficiently cut the tissue.

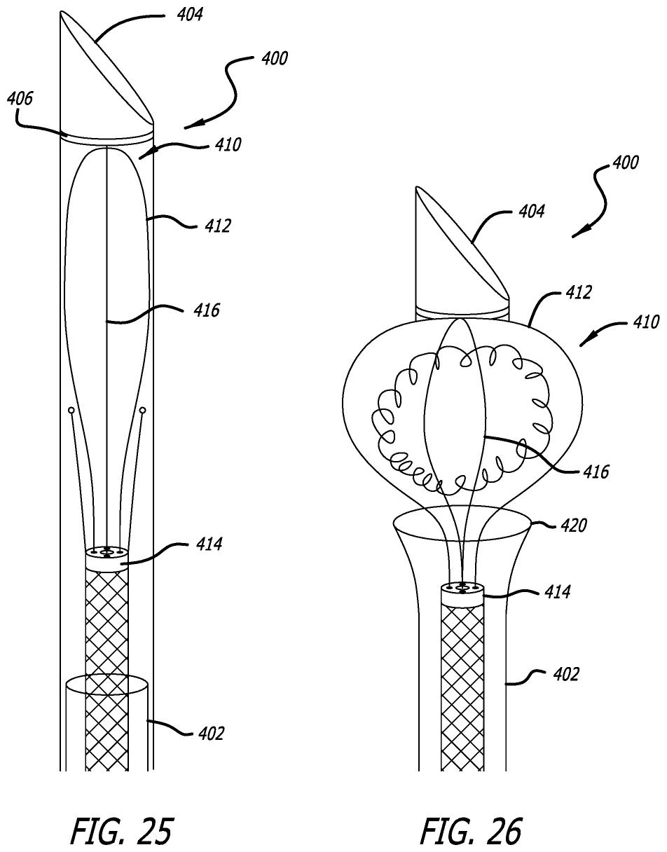

FIGS. 25-28 show an embodiment 400 of a biopsy device that may be ideally suited for retrieving soft, non-fibrous tissue, such as lung tissue. The device 400 generally includes a sheath 402 and a perforating tip 404 that is flush with, but extendable from, the sheath 402. In a retracted position, shown in FIG. 25, the tip 404 is joined with the sheath 402 by a sheath cap 406. The sheath cap 406 combined with the tip 404 allows for easy insertion of the device and caps the payload once the payload is captured and pulled back into the needle to prevent the payload from falling out of the tip of the needle.

Proximal of the tip 404, and contained within the sheath 402, is located an umbrella assembly 410, preferably constructed of nitinol, stainless steel, or similar materials. The umbrella assembly 410 includes a plurality of longitudinal struts 412, which may be coated with a polymer coating. The struts extend from, and are connected to, the tip 404 at a distal end and to a push-pull member 414 at a proximal end of the struts. A center wire 416 extends through a center of the push-pull member and is slidingly disposed therein. The push-pull member 414 extends to a proximal end of the device where it and the center wire 416 may be manipulated by an operator to change the distance between the tip 404 and the push-pull member 414. Shortening the distance causes the struts 412 to flare outwardly, as seen in FIG. 26, and tissue may be trapped on the inside of the device when flared.

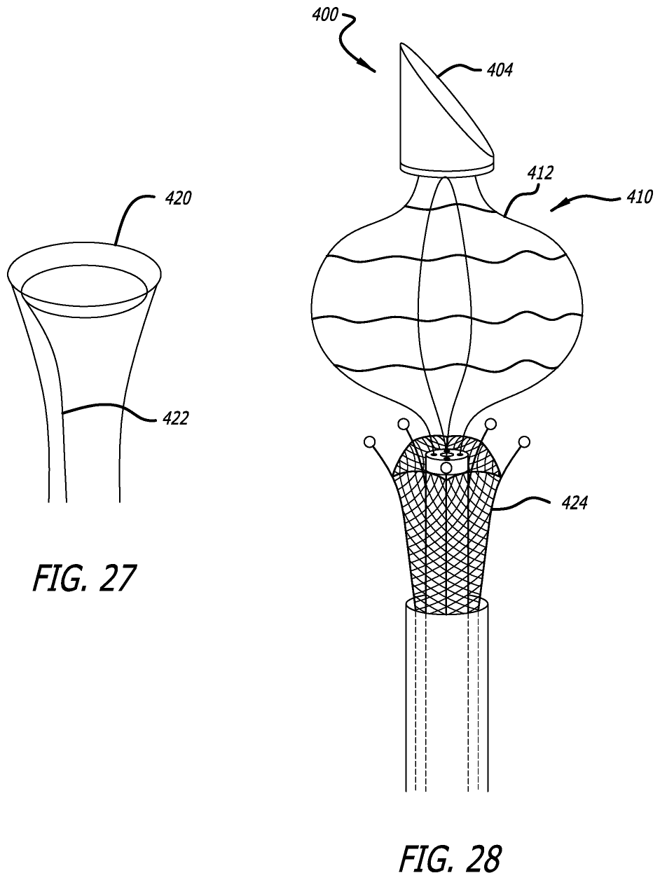

The sheath 402 may have a distal end 420 that is able to be flared to create a funnel, as seen in FIG. 26. Control of the flaring may be optionally facilitated by the use of a lasso mechanism 422 shown in FIG. 27. Alternatively, an inner nitinol mesh receiver 424 may be advanced that automatically flares when exiting a distal end of the sheath 402, as shown in FIG. 28.

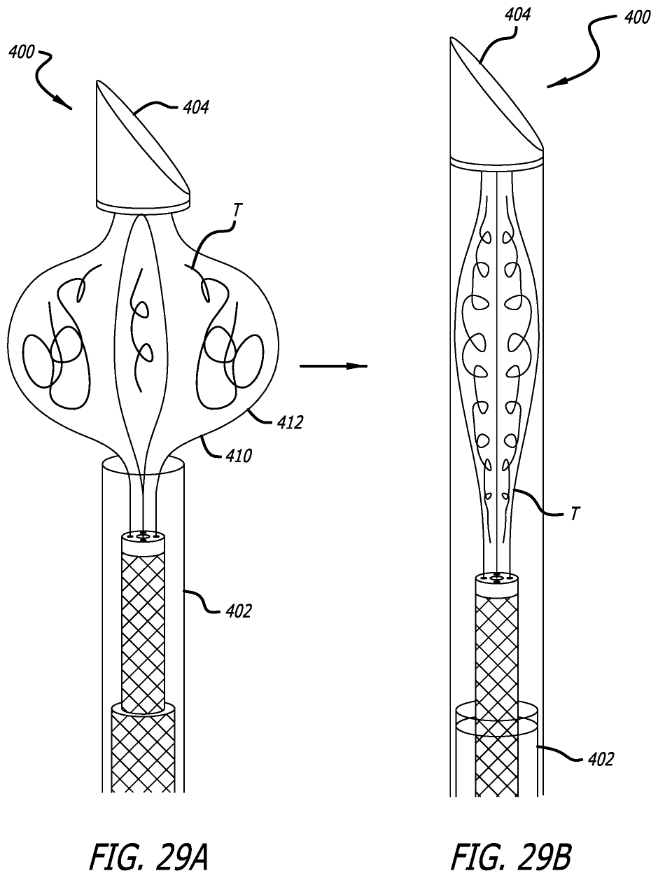

FIG. 29A depicts tissue being captured in the umbrella assembly 410 and FIG. 29B depicts the tissue T being retracted into the sheath 402. It can be seen that the embodiment 400 accomplishes the goal of maximizing the surface area/volume ratio of the payload collecting Nitinol structure to enable the collection of a sufficient amount of lung tissue to run diagnostic tests while minimizing the risks of false negative results.

Navigating to target lesions in the lungs often involves the use of bronchoscopes or electromagnetic navigation bronchoscopy (ENB) systems. Such tools include working channels, which are limited in size in order to maximize navigability. These working channels are typically no greater than 2 mm in diameter. Due to these small working channels, biopsy needles have to be extremely small and, in some cases, are not able to acquire an adequate tissue sample size.

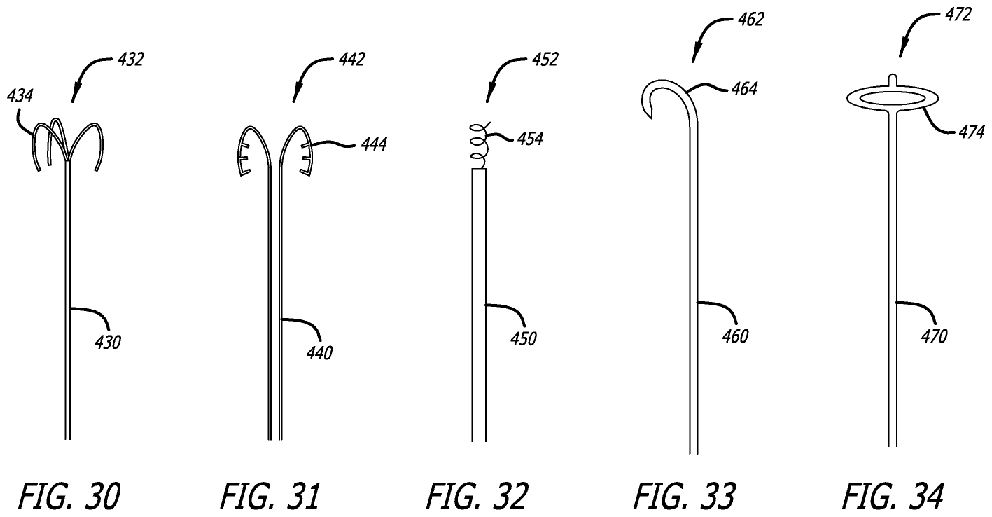

The guidewire concepts of FIGS. 30-34 address this problem by providing an endobronchial exchange method of the invention. The guidewires shown in these figures include a variety of distal anchors that allow the guidewire to be advanced through the working channel of the bronchoscope and attached to a target lesion. Once the guidewire is secured to the lesion, the bronchoscope may be removed while leaving the guidewire in place. Without the distal anchor, the guidewire would not remain in place and a subsequent device would not be assured of reaching the targeted lesion.

The guidewires shown in FIGS. 30-34 are envisioned for use with any or all of the biopsy devices described herein. Additionally, these guidewires may optionally be used with RF energy to help with crossing penetration, coagulation and sealing. RF energy may also be used to ablate the surrounding lesion.

The guidewires of FIGS. 30-34 each have unique distal anchor features that include first and second configurations. The first configurations for each are compressed, axial configurations that are able to be contained and fired from the working channel of a bronchoscope or endoscope. The second configurations are expanded relative to the first configurations and the anchors are biased or heat set to the expanded configurations such that once released from the working channel and deployed into the target tissue, the distal anchors self-expand and grab the tissue sufficiently to remain in place during the endobronchial exchange with a high capacity biopsy device such as those described herein.

FIG. 30 shows a guidewire 430 with a distal anchor 432 that includes a plurality of hooks 434. The hooks may be heat-set or have a spring force that causes the hooks 434 to deploy when exiting the working channel of the bronchoscope.

FIG. 31 shows a guidewire 440 with a distal anchor 442 that includes a plurality of barbed hooks 444. The barbed hooks 444 may be heat-set or have a spring force that causes the hooks 444 to deploy when exiting the working channel of the bronchoscope.

FIG. 32 shows a guidewire 450 with a distal anchor 452 that includes a corkscrew 454. The corkscrew may be heat-set or have a spring force that causes the corkscrew 454 to assume a helical shape when exiting the working channel of the bronchoscope.

FIG. 33 shows a guidewire 460 with a distal anchor 462 that includes a single hook 464. The hook 464 may be heat-set or have a spring force that causes the hook 464 to deploy when exiting the working channel of the bronchoscope.

FIG. 34 shows a guidewire 470 with a distal anchor 472 that includes a set of pinchers 474. The pinchers 474 may be heat-set or have a spring force that causes the pinchers 474 to deploy when exiting the working channel of the bronchoscope.

As stated above, these guidewires can be used with any of the embodiments of biopsy devices described herein. Additionally, embodiments are described below that are specifically designed for use with these anchored guidewires.

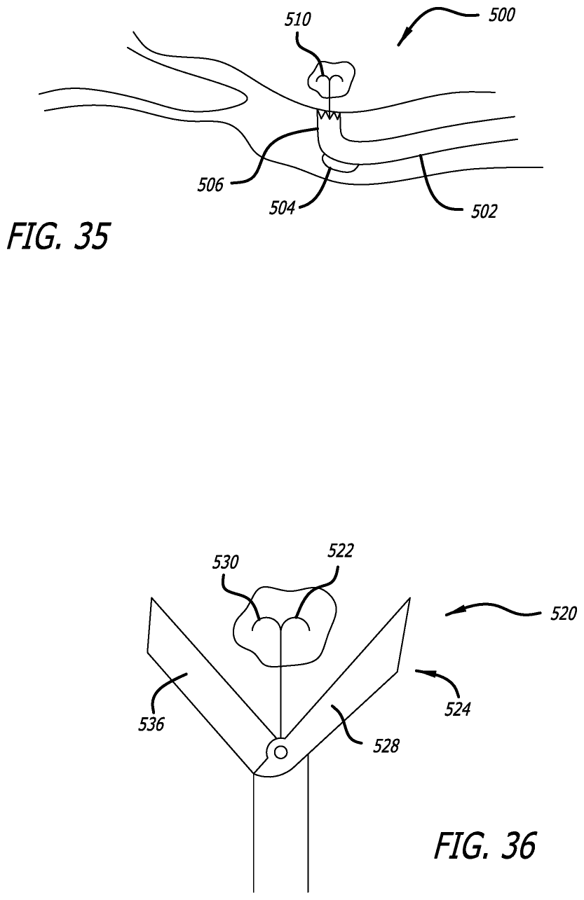

For example, FIG. 35 shows an embodiment 500 that provides a sheath 502 having an external balloon 504 that may be inflated to manipulate the distal end 506 of the sheath in order to angle the distal end 506 toward a lesion adjacent the airway. The distal end of the sheath includes a cutting device that allows the lesion or a sample thereof to be accessed. An anchoring guidewire 510, such as described above, may then be pulled into the sheath 502, pulling the tissue with it. This embodiment 500 may include the application of RF energy to help with crossing penetration, coagulation and sealing, and also to ablate surrounding lesion tissue.

FIG. 36 shows another example of an embodiment 520 of a biopsy tool that is suited to ride over an anchored guidewire 522, such as described above. The device 520 includes a distal cutting tool 524 that includes jaws 526 and 528. Once the distal cutting tool 524 reaches a bronchial wall next to the lesion, the tool may be opened and advanced over the tissue surrounding the anchor 530 of the guidewire 522. The jaws may be opened, closed and rotated repeated to take larger sample sizes. The jaws may also communicate RF energy in combination with varying amounts of clamping force in order to either cut or seal tissue.

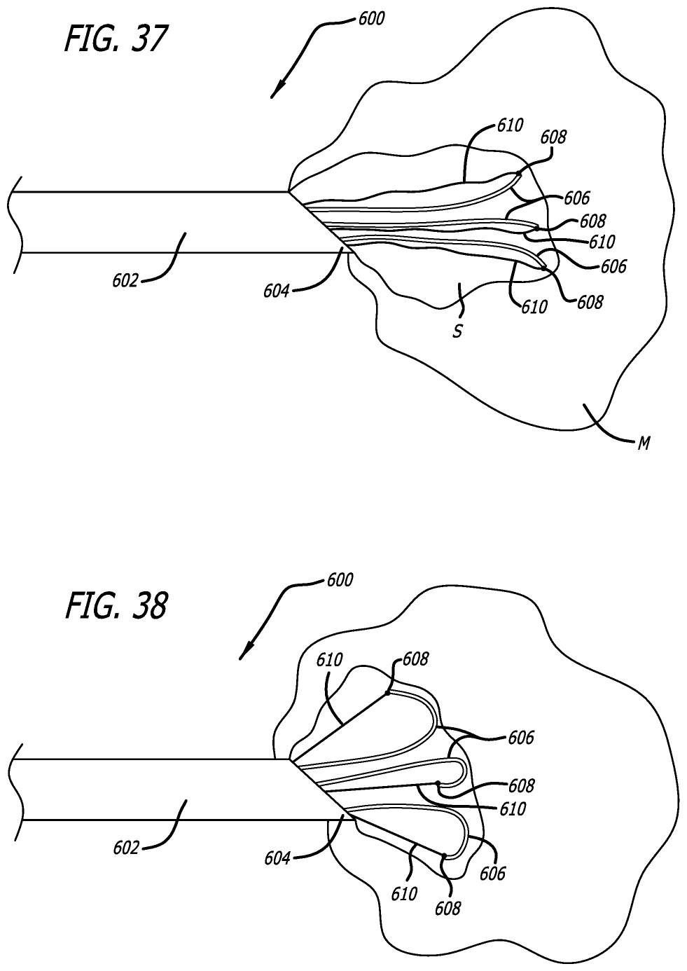

FIGS. 37 and 38 show an example of an embodiment 600 that addresses a concern known as “seeding.” Seeding takes place when a cancerous sample is dragged through healthy tissue, and cancerous cells break off from the sample and “seeds” itself into the healthy tissue, potentially expanding the tumor or developing a new tumor. The embodiment 600 addresses this concern by gripping the distal end of the sample an pulling it into the catheter, as opposed to dragging the sample into the catheter by the proximal end, leaving the distal end to potentially break off.

FIG. 37 shows the device 600 as including a needle or catheter 602, which may have a trocar-shaped tip 604. Deployed from the catheter 602 are at least one, or as shown a plurality of sampling tools 606 in the form of wires or flexible needles. Each of the plurality of sampling tools 606 have a distal end 608 connected to a pull wire 610. The pull wires are routed back into the catheter 602 and may be used to manipulate the flexible sampling tools 606. The device 600 is shown as having been deployed into a mass M, but not yet retracted.

FIG. 38 shows a sample S being retrieved from the mass M. The sample S is retrieved by pulling on the pull wires 610, thus causing the sampling tools 606 to splay outwardly and turn back on themselves, thus forming hooks. The pull wires 610 are pulled until the sampling tools 606 and sample S are completely retracted into the catheter 602.

In order to ensure that the pull wires 610 are cause the sampling tools 606 to splay outwardly, thus maximizing the size of the sample S, the sampling tools 606 may be formed of a memory metal such as Nitinol, and heat set to curve outwardly when deployed.

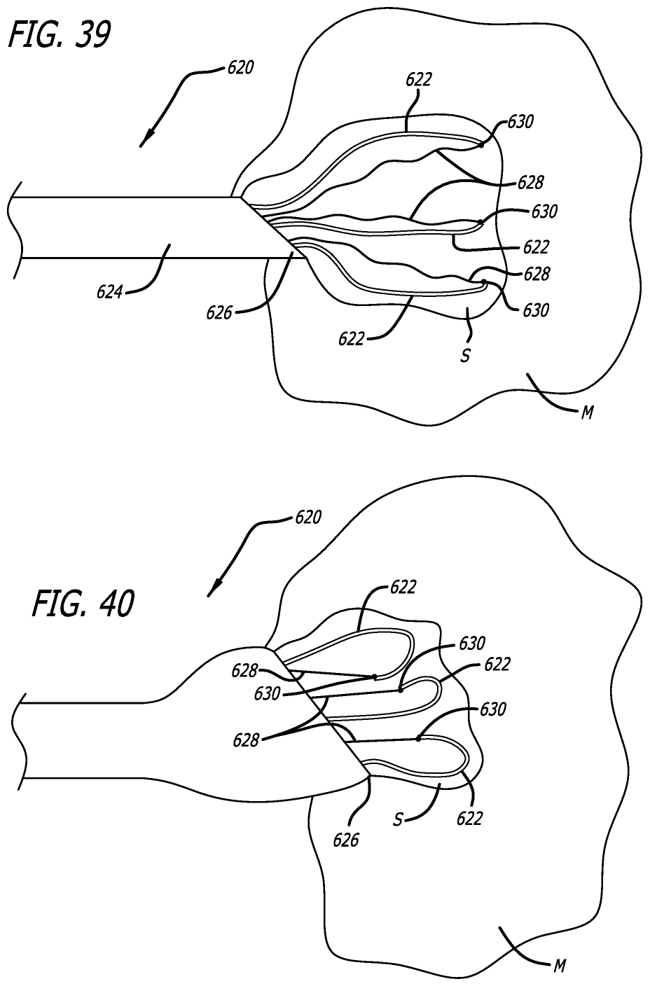

FIGS. 39 and 40 show an alternative embodiment 620 similar to embodiment 600 except that the sampling tools 622 are curved inwardly instead of outwardly. FIG. 39 shows the natural, deployed shape of the tools 622 after being deployed from a catheter 624. The tools 622 can be seen as having a complex curve that expands outwardly from the distal end 626 of the catheter 624, resulting in the pull wires 628 attached to the distal ends 630 of the sampling tools 622 being positioned inwardly of the distal ends 630.

As can be seen in FIG. 40, when the pull wires 628 are retracted, the sampling tools 622 fold inwardly on themselves, capturing the tissue sample S from the mass M and pulling the sample S into the catheter 624.

In some or all of the embodiments shown herein, it may be beneficial to have a distal catheter end that has a lower durometer than the body of the catheter. The durometer may be selected to allow expansion of the distal end of the catheter during sample retrieval while still being able to puncture tissue. In the embodiments shown in which a needle is used in addition to the catheter, an even softer durometer tip may be used to allow greater expansion, resulting in even a larger sample size. FIG. 40 shows an example of a catheter 624 having a distal end or tip 626 of reduced durometer.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A biopsy tool comprising:
a sheath;
a needle disposed within the sheath and having a first configuration and a second configuration;
wherein in the first configuration the needle is radially constrained by the sheath;
wherein in the second configuration the needle includes a tissue engaging feature that may expand such that the tissue engaging feature is located radially outside an outer diameter of the sheath;
wherein further in the second configuration, the tissue engaging features is useable to at least partially remove tissue samples and relocate the tissue samples to an inner lumen of the biopsy tool.

2. The biopsy tool of claim 1 wherein the sheath further comprises a distal edge that is sharpened to assist in completely removing said at least partially removed tissue samples.

3. The biopsy tool of claim 1 wherein said inner lumen is located within said sheath.

4. The biopsy tool of claim 1 wherein said inner lumen is located within said needle.

5. The biopsy tool of claim 1 wherein said tissue engaging feature comprises a blade.

6. The biopsy tool of claim 1 wherein said tissue engaging feature comprises a wireform covered with a film.

7. The biopsy tool of claim 1 wherein said needle comprises a rolled metal sheet that expands when released from said sheath.

8. The biopsy tool of claim 1 wherein said needle comprises a coil.

9. A tissue removal method comprising:
navigating a sheath to a target location;
advancing a tool having a first configuration while housed within the sheath until at least a distal end of the tool emerges from a distal end of the sheath;
changing a state of the tool from said first configuration to a second configuration, said second configuration having a width that is greater than a diameter of the sheath;
using the tool in the second state to remove tissue from the target location;
changing the state of the tool from the second configuration back to the first configuration;
retracting the tool into the sheath with the removed tissue.

10. The tissue removal method of claim 9 wherein changing the state of the tool from the second configuration back to the first configuration and retracting the tool into the sheath with the removed tissue occurs simultaneously.

11. The tissue removal method of claim 9 further comprising applying RF energy to the tissue with the tool.

12. The tissue removal method of claim 9 wherein changing a state of the tool from said first configuration to said second configuration comprises allowing the tool to self-expand once released from the sheath.

13. The tissue removal method of claim 9 wherein changing a state of the tool from said first configuration to said second configuration comprises actively manipulating controls at a proximal end of the sheath.

14. The tissue removal method of claim 9 wherein retracting the tool into the sheath with the removed tissue comprises compressing the tissue into the sheath.

15. The tissue removal method of claim 9 wherein retracting the tool into the sheath with the removed tissue comprises trimming excess tissue with a sharpened distal end of the sheath as the tissue is being pulled into the sheath.

16. A method of taking a biopsy comprising:
navigating an endoscope having a working channel to a location adjacent a targeted tissue area;
advancing a distal end of the guidewire out of a distal end of the working channel and into the targeted tissue area;
deploying an anchor at the distal end of the guidewire;
pulling the endoscope out of the patient while leaving the guidewire in place;
advancing a biopsy tool over the guidewire;
capturing a tissue sample using the biopsy tool;
removing the tissue sample, the biopsy tool, and the guidewire from the patient.

17. The method of claim 16 wherein capturing the tissue sample comprises capturing a tissue sample that surrounds the anchor.

18. The method of claim 16 wherein deploying an anchor at the distal end of the guidewire comprises allowing said anchor to self-expand.

19. The method of claim 16 wherein capturing a tissue sample using the biopsy tool comprises changing a state of the tool from said first configuration to a second configuration, said second configuration having a width that is greater than a diameter of the sheath.

20. The method of claim 19 wherein changing the state of the tool comprises releasing the tool from a sheath.
